(19) United States
(12) Patent Application Publication
(10) Pub. No.: US 2003/0105159 A1
(43) Pub. Date: Jun. 5, 2003

(54) Kavalactone compositions and methods of use

(76) Inventors: Joel McCleary, The Plains, VA (US); Peter S. Staats, Towson, MD (US)

Correspondence Address: FISH & RICHARDSON PC, 225 FRANKLIN ST, BOSTON, MA, 02110, US

(21) Appl. No.: 10/214,624

(22) Filed: Aug. 8, 2002

Related U.S. Application Data
(60) Provisional application No. 60/311,437, filed on Aug. 10, 2001.

(51) Int. Cl. A61K 31/35; A61K 31/366; A61K 35/78; A61K 31/16

(52) U.S. Cl. 514/460; 514/625; 424/760

(57) ABSTRACT

This invention relates to kavalactone-containing compositions, and more particularly to compositions having compounds derived from kavalactones and from capsaicinoids. The compositions are useful in modulating pain, and thus can be used to mediate, or eliminate, sensations of pain, thereby providing pain relief and reduction.

### CROSS-REFERENCE TO RELATED APPLICATIONS

This application claims benefit of U.S. application Ser. No. 60/311,437, filed, Aug. 10, 2001, which is herein incorporated by reference in its entirety.

### BACKGROUND

Oceania (i.e., the Pacific island communities of Micronesia, Melanesia and Polynesia) is an area where kava roots have been highly regarded by native medicine men for its ability to reduce anxiety and stress. In recent years, the Kava plant has been scientifically scrutinized, with certain of its active constituents being identified. The psychoactive ingredients of the Kava root have been identified as kavalactones. A total of sixteen kavalactones have been identified to date, including kawain, dihydrokawain (a.k.a. marindinin), methysticin, dihydromethysticin, yangonin, and desmethoxyyangonin. These compounds are neutral, nitrogen-poor compounds that may be specifically referred to as substituted alpha.-pyrones. The lactone ring is substituted by a methoxy group in the C-4 position, and the compounds vary in their substitution by either a styryl residue (e.g., yangonin, desmethy-oxyyangonin, kawain, and methysticin) or by a phenylethyl residue (e.g., dihydrokawain and dihydromethysticin) at the C-6 position.

The particular kavalactones in a Kava root extract vary depending upon its origin. The concentration ranges of total kavalactone levels in the Kava root extracts employed, e.g., in the US are generally within the range of 10 to 30 wt %. Kava root extract is widely available in the world as an herbal supplement in the form of tablets, capsules, and dragees made of pharmaceutical grade extract. The Kava root extract lactones provide an anxiolytic effect, relieving nervous anxiety, tension, and restlessness, with their efficacy as a relaxant having been demonstrated in clinical studies. The kavalactones also effect muscle relaxation.

Capsaicin, a substance from the solanaceae family, is known to be effective in mediating pain. Capsaicin is known to desensitize nociceptors and various clinical trials have investigated its analgesic effects. It is known to be an effective agent for pain relief and has been utilized to relieve various types of pain, including musculoskeletal pain and neuropathic pain for example. Capsaicin, however, is also known to cause sensations of burning pain, sensation of heat, and hyperalgesia distinct from the neuropathic pain for which relief is sought. As such, patient compliance is low in treatment protocols using capsaicin. It is thus desirable to develop ways in which the irritation, discomfort, and burning pain of the capsaicin can be modulated, while leaving unaffected, the capsaicin's beneficial effect on pain relief.

### SUMMARY

This invention relates to kavalactone-containing compositions, and more particularly to compositions having compounds derived from kavalactones and from capsaicinoids. The compositions are useful in modulating pain, and thus can be used to mediate, or eliminate, sensations of pain, thereby providing pain relief and reduction.

In one embodiment, the invention relates to an analgesic topical composition having: (a) a kavalactone; (b) capsaicinoid or synthetic derivatives thereof; and (c) a pharmaceutically acceptable carrier; wherein the weight ratio of(a):(b) is from 5000:1 to 1:2 (e.g., 800:1 to 1:1; 500:1 to 5:1). In other aspects, the composition includes an effective amount of kavalactones, active kavalactones, or capsaicinoids. In other aspects, the compositions can include one or more kavalactones or active kavalactones, or one or more capsaicinoids.

In another embodiment, the compositions herein can have a kavalactone (e.g., one or more kavalactones, one or more active kavalactones) in about 1-50% (alternatively about 1-10%, alternatively about 10-20%, alternatively about 20-30%, alternatively about 30-40%, alternatively about 40-50%), by weight and a capsaicinoid or its synthetic derivative (e.g., one or more capsaicinoids or derivatives thereof) in about 0.001-4% (alternatively about 0.001-0.01%, alternatively about 0.01-0.1%, alternatively about 0.1-0.5%, alternatively about 0.5-1%, alternatively about 1-2%, alternatively about 2-3%, alternatively about 3-4%), by weight.

In another embodiment, the composition are any of those herein wherein the topical composition includes a kavalactone in about 5-20% by weight and a capsaicinoid or its synthetic derivative in about 0.01-2%, by weight; those wherein the capsaicinoid is 8-methy-N-vanillyl-6-nonenamide, 8-methyl-N-vanillyl-nonamide, or a combination thereof; those wherein the capsaicinoid is N-vanillyl-9-octadecenamide; those wherein the kavalactone is an active kavalactone selected from kawain, dihydrokawain, dihydromethysticin, methysticin, yangonin, desmethoxyyangonin, or a combination thereof; those wherein the kavalactone includes synthetic active kavalactone; and those wherein the capsaicinoid includes synthetic capsaicinoid.

The invention also includes a patch including any of the compositions herein. One embodiment is a patch including a composition-containing material layer, wherein the composition includes a kavalactone and a capsaicinoid or synthetic derivatives thereof; and the patch wherein the kavalactone is an active kavalactone and the capsaicinoid is 8-methy-N-vanillyl-6-nonenamide, 8-methyl-N-vanillyl-nonamide, or a combination thereof.

The invention also relates to a method for providing analgesia in a subject (e.g., humans, animals, mammals) inclduing administering concurrently to the subject in need of such treatment an effective amount of any of the compositions herein, including those having: (a) a kavalactone; (b) a capsaicinoid or synthetic derivatives thereof; and (c) a pharmaceutically acceptable carrier; wherein the weight ratio of (a):(b) is from about 5000:1 to about 1:2; those wherein the kavalactone is an active kavalactone selected from kawain, dihydrokawain, dihydromethysticin, methysticin, yangonin, desmethoxyyangonin, or a combination thereof; those wherein the weight ratio of (a): (b) is from about 800:1 to about 1:1; those wherein the weight ratio of (a): (b) is from about 500:1 to about 5:1; those wherein the topical composition includes a kavalactone in about 1-50%, by weight and a capsaicinoid or its synthetic derivative in about 0.001-4% by weight; those wherein the topical composition includes a kavalactone in about 5-20% by weight and a capsaicinoid or its synthetic derivative in about 0.01-2%, by weight; those wherein the capsaicinoid is 8-methy-N-vanillyl-6-nonenamide, 8-methyl-N-vanillyl-nonamide, or a combination thereof; those wherein the capsaicinoid is N-vanillyl-9-octadecenamide.

The invention also relates to a method for ameliorating the irritation associated with a capsaicinoid including simultaneous topical administration of a kavalactone and a capsaicinoid; and such methods wherein the capsaicinoid is administered to a subject for modulating pain. The methods herein can be those including administration of a topical composition comprising a kavalactone in about 1-50%, by weight and a capsaicinoid or its synthetic derivative in about 0.001-4% by weight, to a subject.

The invention also relates to a method for ameliorating primary and secondary hyperalgesia associated with capsaicin including simultaneous topical administration of a kavalactone and a capsaicinoid; a method for treating intractable myofacial pain (or symptoms thereof) including topical administration of any composition herein to a subject; a method for treating osteoarthritis pain (or symptoms thereof) including topical administration of a composition of any composition herein to a subject; a method for treating neuropathic pain (or symptoms thereof) including topical administration of any composition herein to a subject.

In other embodiments, the invention relates to any composition herein wherein the kavalactone is an active kavalactone (e.g., a kavalactone that has demonstrated physiological activity). In other embodiments, the invention relates to any composition herein wherein the kavalactone is an active kavalactone that is dihydrokawain, dihydromethysticin, kawain, yangonin, methysticin, desmethoxyyangonine, or a combination thereof; or that is S-(+) dihydrokawain, S-dihydromethysticin, S-(+) kawain, yangonin, S-methysticin, desmethoxyyangonine, or a combination thereof.

In other embodiments, the invention relates to any of the compositions herein, wherein the composition is non-ingestible; to any of the compositions herein, wherein the composition is void of cooling carminative agents (e.g., peppermint, menthol, spearmint, carvone); to any of the compositions herein, wherein the composition is for non-oral administration; to any of the compositions herein, wherein the composition is for topical administration; to any of the compositions herein, wherein the composition is void of flavoring agents (e.g., chocolate, vanilla, fruit flavors); to any of the compositions herein, wherein the composition is void of piperidides; to any of the compositions herein, wherein the composition is essentially void (e.g., contains less than 1%, alternatively less than 0.5%, alternatively, less than 0.25%, by weight) of piperidides; to any of the compositions herein, wherein the composition is void of oatmeal-derived materials; to any of the compositions herein, wherein the composition is void of joint support supplements (e.g., boswellin, glucosamine, chondroitin, or methylsulfonylmethane); or to any of the compositions herein, wherein the composition is void of stomach buffering agents (e.g., stevia, glycyrrizinate).

The details of one or more embodiments of the invention are set forth in the accompanying drawings and the description below. Other features, objects, and advantages of the invention will be apparent from the description and drawings, and from the claims.

### DETAILED DESCRIPTION

In one aspect, the invention relates to a medicinal ointment including 1-50% (e.g., about 1-10%, about 10-20%, about 20-30%, about 30-40%, about 40-50%) by weight kavalactone, (e.g., active kavalactone that is kawain, dihydrokawain, dihydromethysticin, methysticin, yangonin, desmethoxyyangonin, or a combination thereof), about 0.001-4% (e.g., about 0.001-0.01%, about 0.01-0.1%, about 0.1-0.5%, about 0.5-1%, about 1-2%, about 2-3%, about 3-4%) capsaicinoid, and a pharmaceutically acceptable carrier. A kavalactone is any lactone-containing compound derived from the kava kava root. The term “active kavalactone” herein refers only to kawain, dihydrokawain, dihydromethysticin, methysticin, yangonin, desmethoxyyangonin, or a combination of them. The amount of kavalactone or active kavalactone can be an effective amount of compound to produce the desired effect (e.g., mediation of irritation, or burning sensation of capsaicin).

Capsaicinoids are compounds derived from an extract of a capsicum from the solanaceae family, including Capsicum frutescens Linne and Capsicum annum Linne, and chemical derivatives thereof. In one embodiment, the capsaicinoids are compounds derived from an extract of a capsicum from the solanaceae family (e.g., capsaicin, dihydrocapsaicin, nordihydrocapsaicin, homocapsaicin, homodihydrocapsaicin). In another embodiment, the capsaicinoids are 8-methy-N-vanillyl-6-nonenamide, 8-methyl-N-vanillyl-nonamide, or a combination thereof. Capsaicinoids can be obtained as an extract from the fruit of plants in the Capsicum genus (e.g., hot pepper, chili pepper, cayenne) or can be of synthetic origin. Capsaicinoids that are derivatives of those compounds found in the plants or plant extracts described above, are made synthetically from natural or synthetic sources using synthetic chemistry reagents and methods known in the art. The amount of capsaicinoid can be an effective amount of compound to produce the desired effect (e.g., modulation, reduction, or relief from pain).

A pharmaceutically acceptable carrier can include mineral oil, liquid petroleum, white petroleum, propylene glycol, polyoxyethylene polyoxypropylene compound, emulsifying wax, water, sorbitan monostearate, polysorbate 60, cetyl esters wax, cetyl alcohol, 2-octyldodecanol, and stearyl alcohol. An acceptable carrier can include water, a solvent, an emollient, a surfactant, a preservative, or a combination thereof. Water, when present, can be in an amount of 5 to 80% by weight. Other than water, the acceptable carrier can also contain a relatively volatile solvent such as a monohydric C1-C3 alkanol (e.g., methyl alcohol or ethyl alcohol) in an amount of 1 to 70% by weight, and an emollient such as those in the form of silicone oils and synthetic esters in an amount of 0.1 to 30% by weight. Other solvents that are acceptable carriers include any suitable for administration of kavalactones and capsaicinoids, for example, dimethyl sulfoxide, C1-C20 alcohols, glycols, and ethers. Anionic, nonionic, or cationic surfactants can also be included in the acceptable carrier. The concentration of total surfactants can be from 0.1 to 40% by weight. Examples of anionic surfactants include soap, alkyl ether sulfate and sulfonate, alkyl sulfate and sulfonate, alkylbenzene sulfonate, alkyl and dialkyl sulfosuccinate, C8-C20 acyl isethionate, acyl glutamate, C8-C20 alkyl ether phosphate, and a combination thereof. Examples of nonionic surfactants include C10-C20 fatty alcohol or acid hydrophobe condensed with from 2 to 100 moles of ethylene oxide or propylene oxide per mole of hydrophobe; C2-C10 alkyl phenol condensed with from 2 to 20 moles of alkylene oxide; mono and di-fatty acid ester of ethylene glycol; fatty acid monoglyceride; sorbitan, mono- and di-C8-C20 fatty acid; block co-polymer (ethylene oxide/propylene oxide); polyoxyethylene sorbitan, and a combination thereof. Preservatives can also be included in the biological acceptable carrier to prevent growth of potentially harmful microorganisms, and can be employed in an amount of 0.01 to 2% by weight. Examples of preservatives include alkyl ester of para-hydroxybenzoic acid, hydantoin derivative, propionate salt, and a variety of quaternary ammonium compounds. Each preservative should be selected based on its compatibility with other ingredients in the composition. An ointment of this invention can be applied to any particular surface area of the body (including the skin, mouth, or gums). EUCERIN crème is a suitable formulation for use in making the compositions herein.

The invention also relates to methods of making any of the compositions herein comprising combining the materials, each added alone, or in combination. The components can be combined or added in a dry or liquid state, and mixed in suitable apparatuses (e.g., drum, bowl, containers) compatible with the components of the composition.

In another aspect, the invention is a patch (see, for example, U.S. Pat. No. 5,186,938) including a kavalactone/capsaicinoid-containing material layer. More specifically, the material layer, e.g., a pad or a pressure-sensitive adhesive, serves as a substrate for receiving about 1-50% (alternatively about 1-10%, alternatively about 10-20%, alternatively about 20-30%, alternatively about 30-40%, alternatively about 40-50%) by weight kavalactone or active kavalactone and about 0.001-4% (alternatively about 0.001-0.01%, alternatively about 0.01-0.1%, alternatively about 0.1-0.5%, alternatively about 0.5-1%, alternatively about 1-2%, alternatively about 2-3%, alternatively about 3-4%) capsaicinioid. The composition can have 1-50% (alternatively about 1-10%, alternatively about 10-20%, alternatively about 20-30%, alternatively about 30-40%, alternatively about 40-50%) by weight kavalactone or active kavalactone, and about 0.001-4% (alternatively about 0.001-0.01%, alternatively about 0.01-0.1%, alternatively about 0.1-0.5%, alternatively about 0.5-1%, alternatively about 1-2%, alternatively about 2-3%, alternatively about 3-4%) capsaicinoid associated with the material layer (e.g., impregnated, embedded, or coated on the surface). A patch optionally has a protective layer intimately adhered to one side of the material layer, which is resistant to passage of the kavalactone or active kavalactone and capsaicinoid. In other embodiments, the invention is a patch including any of the compositions herein, wherein the amounts of kavalactone (e.g., active kavalactone) and capsaicinoids are any of those as delineated herein.

An active kavalactone-containing composition can be administered using an implantable device. Implantable devices and related technology are known in the art and are useful as delivery systems where a continuous, or timed-release delivery of pure kavalactone compounds or compositions delineated herein is desired. Additionally, the implantable device delivery system is useful for targeting specific points of pure kavalactone compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the pure kavalactone compounds and compositions delineated herein. Topical-patches having pure dihydrokawain, dihydromethysticin, kawain, methysticin, yangonin, desmethoxyyangonin, or a combination thereof, or a composition thereof (e.g., kavalactone/capsaicinoid combination) are also included in this invention.

Also within the invention is a patch to deliver active kavalactone. A patch includes a material layer (e.g., polymeric, cloth, gauze, bandage) and the kavalactone/capsaicinoid combination as delineated herein. One side of the material layer can have a protective layer adhered to it to resist passage of kavalactone/capsaicinoid compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing,) on the adhesive or device. Also included are peelable masks that can be formulated by placing the composition as a gel or paste on a protective layer made of a film-forming polymer (e.g., polyvinyl alcohol) and an adhesive promoting polymer (e.g., hydrophobic acrylate or methacrylate polymer, such as Pemulen TR2.RTM. from the B.F. Goodrich Company). Alternatively, a hydrogel composition (see, for example, U.S. Pat. No. 5,961,479 or U.S. Pat. No. 5,306,504) including any one or more of the kavalactone/capsaicinoid combinations can be used.

Another aspect of the invention relates to a packaged product including a container, a composition containing a kavalactone/capsaicinoid compositions herein disposed in the container and a label (e.g., sticker, product insert) with the container and having instructions for application of the kavalactone/capsaicinoid compositions herein for treating a pain disorder, or modulating the irritant effects of capsaicinoids.

The invention also covers a pharmaceutical composition having a pure active kavalactone that is kawain, dihydrokawain, dihydromethysticin, methysticin, yangonin, desmethoxyyangonin, or a combination thereof. The subject can be a human or an animal (e.g., dog, cat). The term “pure” refers to a level of 90% or higher. Pure active kavalactone can be derived from natural (e.g., root extract and purification) or synthetic (e.g., synthesis from natural or synthetic materials) means, or a combination thereof.

A crude extract of the kava roots (obtained using various extraction methods (e.g., simple solvent soak, supercritical fluid extraction)) can be used as the source of kavalactones or active kavalactones for the preparation of a composition of this invention. If desired, the kavalactones or active kavalactones can be further purified by column chromatography. They can also be synthesized from readily available starting materials by conventional chemical methods. See, for example, Kostermans, Reclk. Trav. Chim. Pays-Bas., 70, 79 (1951); Klohs et al., J. Org. Chem., 24, 1829 (1959); Spino, et al. Tetrahedron Lett., 37, 6503 (1996), and references cited in each. The kavalactones or active kavalactones present in a composition can be enriched by addition of those kavalactones (from either natural or synthetic sources). The kavalactones or active kavalactones contain one or more asymmetric centers and thus can occur as racemates and racemic mixtures, single enantiomers, individual diastereomers and diastereomeric mixtures. They can also occur in cis- or trans- or E- or Z-double bond isomeric forms.

The compositions herein are useful for ameliorating the burning or irritation (e.g., primary and secondary hyperalgesic effects) associated with capsaicinoids and their use. As such, the compositions allow for more wide-spread use and improved patient compliance of treatment regimens involving capsaicinoids. The compositions herein are useful in treating (e.g., relieving, reducing, modulating) pain (e.g., neuropathic, inflammatory, myofacial, osteoarthritic), pain sensation, or symptoms thereof. In certain instances, as illustrated in the examples herein, the treatment protocols using the compositions herein provided relief in cases where multiple surgical interventions, and multiple medication regimens had previously given unsatisfactory results (e.g., insufficient relief from pain or pain symptoms) or had failed.

In addition to the masking effect against hyperalgesia of the kavalactone-capsaicinoid combination compositions delineated herein, the compositions have an additional benefit in that the undesirable pain or irritation effects attributable to capsaicin are modulated, while the efficacy effects of the capsaicin (e.g., pain relief, pain, reduction, analgesic effect) are retained, or relatively unaffected as compared to capsaicin compositions without the kavalactones. The retention of analgesic effect can be measured using a variety of standard analgesia protocols, including, for example, the formalin test as essentially reported by Tjolsen, et al., Pain, 51, 13 (1992). Depending on the dosages employed, the kavalactones may either potentiate the degree of analgesia beyond that obtainable using capsaicinoids alone, or it may induce analgesia at dosages where no analgesic effect is obtained from either component alone.

All references cited herein, whether in print, electronic, computer readable storage media or other form, are expressly incorporated by reference in their entirety, including but not limited to, abstracts, articles, journals, publications, texts, treatises, technical data sheets, internet web sites, databases, patents, patent applications, and patent publications.

Embodiments are further described in the following representative examples, which do not limit the scope of the invention described in the claims.

### EXAMPLES

### Example 1

A 1% capsaicin cream was prepared by mixing of 455 g of EUCERIN crème with 10 ml of EtOH solution and 5 g natural capsaicin (trans-8-methyl-N-vanilyl-6-noneamide) (purchased from Aldrich Chemical Company, Inc., Milwaukee, Wis.).

### Example 2

A 20% kava+1% capsaicin crème was prepared as follows: 120 g of kava extract paste (84% kavalactones, purchased from Cosmopolitan Trading, Seattle, Wash.) was converted to a homogeneous solution with the aid of 20 ml of EtOH at 70 C. Five grams (5 g) of capsaicin was added to the solution. The solution was mixed with 395 g of EUCERIN crème and left overnight at room temperature to remove excess EtOH.

### Example 3

A Chinese male (40 years old) received 5 g of 1% capsaicin crème on his right shin and 20% kava+1% capsaicin crème on his left shin in a circular motion in a 5 cm diameter, respectively and the applied area was covered with a bandage, then the degree of hyperalgesia was monitored. After 5-10 min, the subject experienced significant pain on his right shin where 1% capsaicin had been applied. In contrast, he did not feel pain on his left shin where 20% kava and 1% capsaicin had been applied. This effect lasted until the applied crème was removed.

### Example 4

The same experiment as in Example 3 was been conducted using three other subjects consisting of two Caucasian males (45 years old and 40 years old) and one Japanese male (48 years old). Significant masking effect of hyperalgesis caused by capsaicin was observed in all subjects.

### Example 5

Capsaicin was applied topically at a concentration of 1% in one extremity. A second extremity had topically applied 1% capsaicin plus 30% kava cream. Both samples were blinded to the subjects. Ten subjects had both mixtures applied. In all subjects the burning associated with 1% capsaicin was blunted (cut on average 60-80%). Four subjects experienced no burning with capsaicin. That evening secondary hyperalgesia was measured in several subjects and found to be markedly reduced in the subjects receiving kava plus capsaicin compositions.

### Example 6

Four subjects were exposed to topical capsaicin at 1% in two extremities. After approximately 1 hour, when the burning became quite intense, either placebo or 30% kava was applied to the effective area in a blinded fashion. All subjects reported a marked reduction in the burning associated with capsaicin in the side receiving capsaicin but not in the side receiving the placebo. This indicates that kava (e.g., kavalactones) were able to counteract the burning (secondary and primary hyperalgesia) associated with capsaicin.

### Example 7

A patient with intractable neuropathic and myofascial pain had topically applied 1% capsaicin plus 30% kava cream. She reported marked reduction in pain in the area applied. This effect lasted for 24 hours. This had previously failed multiple surgical interventions, multiple medications. She also reported a pleasant heat, with no burning associated with capsaicin.

A number of embodiments of the invention have been described. Nevertheless, it will be understood that various modifications may be made without departing from the spirit and scope of the invention. Accordingly, other embodiments are within the scope of the following claims.

What is claimed is:

1. An analgesic topical composition comprising: (a) one or more kavalactones; (b) one or more capsaicinoid or synthetic derivatives thereof; and (c) a pharmaceutically acceptable carrier; wherein the weight ratio of (a):(b) is from 5000:1 to 1:2.

2. The composition according to claim 1, wherein the weight ratio of (a): (b) is from 800:1 to 1:1.

3. The composition according to claim 1, wherein the weight ratio of (a): (b) is from 500:1 to 5:1.

4. The composition according to claim 1, wherein the topical composition comprises a kavalactone in about 1-50% (alternatively about 1-10%, alternatively about 10-20%, alternatively about 20-30%, alternatively about 30-40%, alternatively about 40-50%), by weight and a capsaicinoid or its synthetic derivative in about 0.001-4% (alternatively about 0.001-0.01%, alternatively about 0.01-0.1%, alternatively about 0.1-0.5%, alternatively about 0.5-1%, alternatively about 1-2%, alternatively about 2-3%, alternatively about 3-4%), by weight.

5. The composition according to claim 1, wherein the topical composition comprises a kavalactone in about 5-20% by weight and a capsaicinoid or its synthetic derivative in about 0.01-2%, by weight.

6. The composition according to claim 1, wherein the capsaicinoid is 8-methy-N-vanillyl-6-nonenamide, 8-methyl-N-vanillyl-nonamide, or a combination thereof.

7. The composition according to claim 1, wherein the capsaicinoid is N-vanillyl-9-octadecenamide.

8. The composition according to claim 1, wherein the kavalactone is an active kavalactone selected from kawain, dihydrokawain, dihydromethysticin, methysticin, yangonin, desmethoxyyangonin, or a combination thereof.

9. The composition according to claim 1, wherein the kavalactone includes synthetic active kavalactone.

10. The composition according to claim 1, wherein the capsaicinoid includes synthetic capsaicinoid.

11. A patch including a composition-containing material layer, wherein the composition comprises a kavalactone and a capsaicinoid or synthetic derivatives thereof.

12. The patch according to claim 11, wherein the kavalactone is an active kavalactone and the capsaicinoid is 8-methy-N-vanillyl-6-nonenamide, 8-methyl-N-vanillyl-nonamide, or a combination thereof.

13. A method for providing analgesia in a subject comprising administering concurrently to the subject in need of such treatment an effective amount of: (a) a kavalactone; (b) a capsaicinoid or synthetic derivatives thereof; and (c) a pharmaceutically acceptable carrier; wherein the weight ratio of (a):(b) is from about 5000:1 to about 1:2.

14. The method of claim 13, wherein the kavalactone is an active kavalactone selected from kawain, dihydrokawain, dihydromethysticin, methysticin, yangonin, desmethoxyyangonin, or a combination thereof.

15. The method according to claim 13, wherein the weight ratio of (a):(b) is from about 800:1 to about 1:1.

16. The method according to claim 13, wherein the weight ratio of (a):(b) is from about 500:1 to about 5:1.

17. The method according to claim 13, wherein the topical composition comprises a kavalactone in about 1-50%, by weight and a capsaicinoid or its synthetic derivative in about 0.001-4% by weight.

18. The method according to claim 13, wherein the topical composition comprises a kavalactone in about 5-20% by weight and a capsaicinoid or its synthetic derivative in about 0.01-2%, by weight.

19. The method according to claim 13, wherein the capsaicinoid is 8-methy-N-vanillyl-6-nonenamide, 8-methyl-N-vanillyl-nonamide, or a combination thereof.

20. The method according to claim 13, wherein the capsaicinoid is N-vanillyl-9-octadecenamide.

21. A method for ameliorating the irritation associated with a capsaicinoid comprising simultaneous topical administration of a kavalactone and a capsaicinoid.

22. The method of claim 21, wherein the capsaicinoid is administered to a subject for modulating pain.

23. The method according to claim 21, comprising administration of a topical composition comprising a kavalactone in about 1-50%, by weight and a capsaicinoid or its synthetic derivative in about 0.001-4% by weight, to a subject.

24. A method for ameliorating primary and secondary hyperalgesia associated with capsaicin comprising simultaneous topical administration of a kavalactone and a capsaicinoid.

25. A method for treating intractable myofacial pain comprising topical administration of a composition of claim 1 to a subject.

26. A method for treating osteoarthritis pain comprising topical administration of a composition of claim 1 to a subject.

27. A method for treating neuropathic pain comprising topical administration of a composition of claim 1 to a subject.
